(19) United States
(12) Patent Application Publication
(10) Pub. No.: US 2020/0337590 A1
(43) Pub. Date: Oct. 29, 2020

(54) ASSISTANCE IN THE TREATMENT OF CARDIAC INSUFFICIENCY

(71) Applicant: Bayer Aktiengesellschaft, Leverkusen (DE)

(72) Inventors: Frank KRAMER, Wülfrath (DE); Wilfried DINH, Rhede (DE); Daniel FORLER, Wellesley, MA (US); Hubert TRÜBEL, Wuppertal (DE)

(73) Assignee: Bayer Aktiengesellschaft, Leverkusen (DE)

(21) Appl. No.: 16/961,902

(22) PCT Filed: Jan. 10, 2019
(86) PCT No.: PCT/EP2019/050512

§ 371 Date: Jul. 13, 2020

(30) Foreign Application Priority Data
Jan. 16, 2018 (EP) 18151760.8

(51) Int. Cl. A61B 5/053; G16H 50/20; G16H 20/13

(52) U.S. Cl. CPC A61B 5/0537; G16H 20/13; G16H 50/20

(57) ABSTRACT

The present invention relates to the treatment of cardiac insufficiency. A sensor for measuring the tissue fluid content in the region of the rib cage is implanted in a person suffering from cardiac insufficiency. If the tissue fluid content does not get back to normal within a defined period of time and under treatment with the available standard therapy, treatment with a V1a/V2 vasopessin receptor antagonist is recommended. Dosage of the V1a/V2 vasopressin receptor antagonist and duration of the treatment with the V1a/V2 vasopressin receptor antagonist can be determined on the basis of the acquired sensor data.

### CROSS REFERENCE TO RELATED APPLICATIONS

This application is a national stage application under 35 U.S.C. § 371 of International Application No. PCT/EP2019/050512, filed internationally on Jan. 10, 2019, which claims the benefit of European Application No. 18151760.8, filed Jan. 16, 2018.

### FIELD OF THE DISCLOSURE

The present invention relates generally to the treatment of heart failure and in particular, a system including a sensor to assist in monitoring a patient at risk of heart failure.

### SUMMARY OF THE DISCLOSURE

In some embodiments, to assist in the treatment of cardiac insufficiency, a sensor for determining the tissue water content in the region of the chest is used for a person who is suffering from symptomatic heart failure. If the water content does not normalize within a defined period under treatment with the available standard therapy, a treatment with a V1a/V2 vasopressin receptor antagonist is recommended. The dosage of the V1a/V2 vasopressin receptor antagonist and the duration of treatment with the V1a/V2 vasopressin receptor antagonist can be specified on the basis of the sensor data.

In the case of a heart failure (HF), the heart is no longer capable of supplying sufficient oxygen and nutrients to the tissues of the body. The removal of metabolic waste products and carbon dioxide is disrupted too.

Different complaints can occur depending on the severity and nature of the heart failure: for example, shortness of breath when climbing stairs or with other physical strains—also even at rest at an advanced stage. Moreover, water retention (edemas) is possible in the case a heart failure, for example in the lungs or the legs or on the back of the foot.

Owing to the decrease in cardiac output in the course of heart failure, the organs are inadequately perfused. Compensatory mechanisms to ensure the supply of oxygen-rich blood to the organs are activated in response to this. These include the increased release of hormones such as epinephrine and norepinephrine, which increase the strength of the heart and increase blood pressure by vascular narrowing. The effector hormones of the renin-angiotensin-aldosterone system lead to an increase in blood volume and also to the increase in blood pressure.

Patients with chronic heart failure exhibit increased serum vasopressin levels, the concentration of which correlates with the severity of the disease. Said increased vasopressin levels worsen the patient's prognosis owing to a V1a receptor-mediated vasoconstriction and a V2 receptor-associated renal water retention. Besides the water retention mediated by V2 receptors and the associated unfavorable hemodynamic consequences in the sense of an increase in afterload, what are also adversely affected are the emptying of the left ventricle, the pressure in the pulmonary vessels and cardiac performance as a result of V1a-mediated vasoconstriction. Furthermore, from animal experimental data, vasopressin is also thought to have a direct hypertrophy-promoting effect on cardiac muscle. In contrast to the renal effect of volume expansion that is mediated by activation of V2 receptors, the direct effect on cardiac muscle is triggered by activation of V1a receptors.

Blocking of the vasopressin receptors is therefore a pathophysiologically appropriate therapeutic approach for heart failure.

Substances which inhibit the action of vasopressin on the V2 receptor and/or on the V1a receptor appear to be suitable for treating heart failure. Especially substances or mixtures of substances having combined activity on both vasopressin receptors (V1a and V2) should bring about desirable renal and hemodynamic effects and thus provide a particularly ideal profile for the treatment of heart-failure patients.

In a first aspect, the present invention provides a V1a/V2 vasopressin receptor antagonist for use in a method for treating a heart failure in a person wearing a sensor for determining the tissue water content in the person's body in the region of the chest, the sensor-measured water content not falling below a defined critical tissue water content value within a defined recovery time span under applied standard therapy.

The present invention further provides a V1a/V2 vasopressin receptor antagonist for use in a method for treating a heart failure in a person wearing a sensor for determining the tissue water content in the person's body in the region of the chest, wherein the dosage of the V1a/V2 vasopressin receptor antagonist and/or the duration of treatment with the V1a/V2 vasopressin receptor antagonist are specified on the basis of the tissue water content.

The present invention further provides a V1a/V2 vasopressin receptor antagonist for use in a method for treating a heart failure in a person wearing a sensor for determining the tissue water content in the person's body in the region of the chest, wherein the dosage of the V1a/V2 vasopressin receptor antagonist and/or the duration of treatment with the V1a/V2 vasopressin receptor antagonist are attuned to the tissue water content over the course of the treatment.

The present invention further provides a system comprising
a sensor for determining the tissue water content in the body of a person in the region of the chest and
a transmitter for transmitting sensor data to a receiver and
the receiver for receiving the sensor data transmitted by the transmitter and
a dosage determination unit for specifying a dosage of a V1a/V2 vasopressin receptor antagonist for the person on the basis of the sensor data and/or
a treatment-duration determination unit for specifying the duration of a therapy with the V1a/V2 vasopressin receptor antagonist.

The present invention further provides a computer program product comprising a data carrier on which there is stored a computer program which can be loaded into the memory of a computer system, where it causes the computer system to execute the following steps:
receiving sensor data from a sensor for determining a tissue water content in the chest region of a person and
ascertaining a dosage of a V1a/V2 vasopressin receptor antagonist on the basis of the sensor data and/or
ascertaining a duration of treatment with a V1a/V2 vasopressin receptor antagonist on the basis of the sensor data.

The present invention further provides a kit comprising the computer program product according to the invention and a medicament containing multiple doses of a V1a/V2 vasopressin receptor antagonist.

The present invention further provides a method comprising the steps of:
providing a sensor for determining a tissue water content in the region of the chest of a person and
capturing data relating to the tissue water content in the region of the chest of the person, the person suffering from heart failure, and
ascertaining a dosage of a V1a/V2 vasopressin receptor antagonist on the basis of the data relating to the water content and/or
ascertaining a duration of treatment with a V1a/V2 vasopressin receptor antagonist on the basis of the sensor data.

The invention will be more particularly elucidated below without distinguishing between the subjects of the invention. On the contrary, the following elucidations are intended to apply analogously to all the subjects of the invention, irrespective of in which context they occur.

### BRIEF DESCRIPTION OF THE DRAWINGS

The present disclosure will now be described, by way of example only, with reference to the accompanying drawings, in which:

FIG. 1 illustrates a block diagram of a system for assisting in the treatment of cardiac insufficiency, according to some embodiments;

FIG. 2 illustrates a block diagram of a system for assisting in the treatment of cardiac insufficiency, according to some embodiments;

FIG. 3 illustrates a block diagram of a system for assisting in the treatment of cardiac insufficiency, according to some embodiments;

FIG. 4 illustrates a flowchart of a method for assisting in the treatment of cardiac insufficiency, according to some embodiments;

FIG. 5 illustrates a flowchart of a method for assisting in the treatment of cardiac insufficiency, according to some embodiments; and

FIG. 6 illustrates a flowchart of a method for assisting in the treatment of cardiac insufficiency, according to some embodiments.

### DETAILED DESCRIPTION

The present invention uses a sensor for determining the tissue water content in the region of the chest of a person in order to initiate a therapy with a V1a/V2 vasopressin receptor antagonist and/or to specify the dosage of the V1a/V2 vasopressin receptor antagonist and/or to end the successful therapy with a V1a/V2 vasopressin receptor antagonist and/or to specify the duration of treatment with a V1a/V2 vasopressin receptor antagonist.

A sensor is a technical component which can capture certain physical or chemical properties and/or the material nature of its environment in a qualitative manner or in a quantitative manner as a measurement variable. The captured variable is captured by means of physical or chemical effects and usually transformed into a further processable electrical signal. Terms used synonymously with the term “sensor” are detector, (measurement-variable) pick-up element or (measurement) pick-up element, and (measurement) sensing element.

The sensor used according to the invention directly or indirectly captures the tissue water content in the body of a person in the region of the chest (thorax).

The sensor can capture one or more values of a variable correlating with the tissue water content in the region of the chest of a person. “Correlation” means that the tissue water content in the region of the chest can in principle be calculated from the corresponding variable (after a calibration if necessary). The correlation can be positive (as values of the measured variable increase, the tissue water content also rises) and negative (as values of the measured variable increase, the tissue water content falls).

The sensor is preferably permanently worn on the person's body (e.g., as a so-called “wearable sensor”) or in the person's body (e.g., as a so-called “implantable sensor”). However, it is also conceivable that the sensor is not continuously with a person, but is used at defined time points by the person suffering from heart failure.

Preferably, the sensor (directly or indirectly) measures the water content continuously. Generally, an individual measurement requires a certain time span. The term “continuously” means that the sensor carries out a multiplicity of individual measurements over a monitoring period generally stretching over several days to weeks, the time interval between two successive individual measurements being sufficiently small for a development of the measured variable over time to be identifiable and for an interpolation to regions between the individual measurements to be possible (in contrast to carrying out measurements at larger time intervals, which merely represent snapshots from which it is, however, not possible to draw any reliable conclusions about the course of the variable between the snapshots).

In a preferred embodiment, the tissue water content is measured via (intrathoracic or transthoracic) impedance. Sensors for measuring intrathoracic or transthoracic impedance are commercially available. There are implantable sensors and sensors in which band electrodes are worn on the skin. Details on measuring intrathoracic or transthoracic impedance and determining the tissue water content in the region of the chest are described in the literature (see, for example: F. Amberger, St. Jude Medical GmbH: Therapie der Herzinsuizienz durch aktive Implantate—Status und Perspektiven [Heart failure therapy through active implants—status and perspectives], KARDIOTECHNIK March 2011, pages 77 bis 81; A. Fein et al.: Evaluation of Transthoracic Electrical Impedance in the Diagnosis of Pulmonary Edema, http://assets.fluke.com/BiomedDocs/PPP085_Impedance_Educational.ppt; Alberto García Lledó et al.: SYSTEM FOR MEASURING THE TRANSTHORACIC ELECTRICAL IMPEDANCE TO THE ECG SIGNAL, INTERNATIONAL CONGRESS ON COMPUTATIONAL BIOENGINEERING; M. Doblaré, M. Cerrolaza and H. Rodrigues (Eds.), España, 2003; W. H. Tand et al: Measuring impedance in congestive heart failure: Current options and clinical applications, Am. Heart. J. 2009 March; 157(3): 402-411).

However, it is also conceivable to use other sensors for determining water content (see, for example: Julian Lenk: Methodenvergleich zur Messung der Körperzusammensetzung bei Patienten mit chronischer Herzinsuffizienz [Comparison of methods for measuring body composition in chronic heart failure patients], thesis for attainment of the academic degree Doctor medicinae submitted to the medical faculty of Charité—Universitatsmedizin Berlin, doctorate obtained on: May 30, 2015).

The tissue water content in the region of the chest of a person is tracked over a relatively long period, i.e., over a period of several days to several weeks.

The tissue water content and/or changes in the tissue water content in the region of the chest is/are used as biomarkers for the start of a therapy with a V1a/V2 vasopressin receptor antagonist and/or for ascertaining the dosage of a V1a/V2 vasopressin receptor antagonist and/or for determining the duration of therapy.

It is, for example, conceivable that a person who is suffering from heart failure is being subjected to a therapy. Therapeutic measures for treating heart failure are, for example, described in: M. Böhm: Herzinsuffizienz [Heart failure], Thieme-Verlag 2000, ISBN 3-13-117151-0. These treatment methods can be referred to as standard therapies. In particular, any therapeutic measure for treating heart failure other than the administration of a V1a/V2 vasopressin receptor antagonist is referred to as standard therapy in this description. The standard therapy is monitored by means of a sensor for determining the tissue water content in the region of the chest. If the therapeutic measures do not lead to a fall in the tissue water content, a therapy with a V1a/V2 vasopressin receptor antagonist is initiated.

The therapy with a V1a/V2 vasopressin receptor antagonist is initiated especially when the increased tissue water content in the chest region as a result of heart failure does not fall below a defined value (critical tissue water content value). The critical tissue water content value can, for example, be specified (defined) by a physician. Preferably, specifying (defining) the critical tissue water content value for each individual patient is preferably done in an automatic and specific manner on the basis of patient data. Examples of patient data which can be used for specifying a critical tissue water content value are: sex, age, weight, weight change, height, severity of heart failure (e.g., in the form of the New York Heart Association (NYHA) classification into classes NYHA I to IV), anamnesis, type and duration and amount of medicaments already taken, blood pressure, central venous pressure, respiratory rate, dyspnea, comorbidities, tissue water content in the chest region and the like.

Patient data can be input into a database, possibly via a network, by a physician and/or care personnel and/or the patient himself/herself and/or relatives. However, they can also be captured automatically by one or more sensors.

The critical tissue water content value does not necessarily have to be a value for the tissue water content in the chest region; it is also conceivable that the value is the value of a variable which correlates with the tissue water content in the chest region. “Correlation” means that the tissue water content in the chest region can in principle be calculated from the corresponding variable (after a calibration if necessary).

The falling of the tissue water content below the critical tissue water content value should occur within a defined time span (recovery time span). The recovery time span can, for example, be specified (defined) by a physician. Preferably, specifying (defining) the recovery time span for each individual patient is preferably done in an automatic and specific manner on the basis of patient data. Examples of patient data which can be used for specifying a recovery time span are: sex, age, weight, weight change, height, severity of heart failure (e.g., in the form of a classification into classes NYHA I to IV), anamnesis, type and duration and amount of medicaments already taken, blood pressure, central venous pressure, respiratory rate, dyspnea, comorbidities, tissue water content value in the chest region and the like.

It is conceivable that the critical tissue water content value and/or the recovery time span are not fixed values, i.e., values which are unchangeable over time, but are preferably automatically attuned to the patient's health status over the course of time.

In a preferred embodiment, the critical tissue water content value and/or the recovery time span are automatically ascertained on the basis of patient data and/or automatically attuned over the course of the disease on the basis of patient data.

If the recovery time span is exceeded and the value for the tissue water content is still above the critical tissue water content value, this indicates that the therapeutic measures taken so far (applied standard therapy) are not resulting in the desired stabilization of the person who is suffering from heart failure. In such a case, a therapy with a V1a/V2 vasopressin receptor antagonist is recommended/initiated.

If a therapy with a V1a/V2 vasopressin receptor antagonist is recommended or initiated, the duration of the treatment with the V1a/V2 vasopressin receptor antagonist can preferably be automatically specified on the basis of the sensor data. Preferably, specifying (defining) the duration of treatment for each individual patient is additionally done on the basis of further person-specific patient data. Examples of patient data which can be used for specifying a duration of treatment are: sex, age, weight, weight change, height, severity of heart failure (e.g., in the form of a classification into classes NYHA I to IV), anamnesis, type and duration and amount of medicaments already taken, blood pressure, central venous pressure, respiratory rate, dyspnea, comorbidities, tissue water content value in the chest region and the like.

It is conceivable that the duration of the treatment with a V1a/V2 vasopressin receptor antagonist is not a fixed value, i.e., a value which is unchangeable over time, but is preferably automatically attuned to the patient's health status over the course of time.

In one embodiment of the present invention, the dosage of the V1a/V2 vasopressin receptor antagonist is automatically specified on the basis of the measured tissue water content in the region of the chest of the person.

The term “dosage” is understood to mean the specification of the medicament to be administered, the particular amount of the medicament (e.g., one tablet or 2 g or 1 mL or the like) and also the frequency of administration of medicament (e.g., once a day or before each meal or the like).

Preferably, specifying (defining) the dosage for each individual patient is preferably done in an automatic manner on the basis of person-specific patient data. Examples of patient data which can be used for specifying a dosage are: sex, age, weight, weight change, height, severity of heart failure (e.g., in the form of a classification into classes NYHA I to IV), anamnesis, type and duration and amount of medicaments already taken, blood pressure, central venous pressure, respiratory rate, dyspnea, comorbidities, tissue water content value in the chest region and the like.

It is conceivable that the dosage with a V1a/V2 vasopressin receptor antagonist is not a fixed value, i.e., a value which is unchangeable over time, but is preferably automatically attuned to the patient's health status over the course of time.

It is conceivable that the ending of the therapy with a V1a/V2 vasopressin receptor antagonist is preferably automatically determined on the basis of the measured tissue water content in the region of the chest of the person. For example, it is conceivable that the tissue water content in the region of the chest falls into a range defined as the normal range. If the tissue water content reaches the normal range, the dosage is set to zero and the therapy with the V1a/V2 vasopressin receptor antagonist is ended.

Preferably, specifying (defining) the normal range for each individual patient is done on the basis of person-specific patient data and preferably in an automatic manner. Examples of patient data which can be used for specifying a dosage are: sex, age, weight, weight change, height, severity of heart failure (e.g., in the form of a classification into classes NYHA I to IV), anamnesis, type and duration and amount of medicaments already taken, blood pressure, central venous pressure, respiratory rate, dyspnea, comorbidities, tissue water content value in the chest region and the like.

It is conceivable that the normal range is not a fixed range of values, i.e., a range of values which is unchangeable over time, but is preferably automatically attuned to the patient's health status over the course of time.

The V1a/V2 vasopressin receptor antagonist can be a substance (an active ingredient) which addresses both the V1a vasopressin receptor and the V2 vasopressin receptor (dual V1a/V2 vasopressin receptor antagonist); however, another possibility is a mixture or a multicomponent system comprising two or more substances (active ingredients), one substance (one active ingredient) of which addresses the V1a vasopressin receptor and another substance (another active ingredient) of which addresses the V2 vasopressin receptor. Preferably, a dual V1a/V2 vasopressin receptor antagonist is concerned.

Examples of V1a/V2 vasopressin receptor antagonists are described in: WO07134862, WO16071212, WO9944613, WO0247679, WO08036755, WO08036759, WO09117144, WO10042714. Preference is given to the V1a/V2 vasopressin receptor antagonist with the designation BR 6819 (https://adisinsight.springer.com/drugs/800050780).

The system according to the invention comprises those components which are at least necessary for technically implementing the invention. The system according to the invention comprises at least one sensor for determining the tissue water content in the body of a person in the region of the chest and a transmitter for transmitting sensor data to a receiver and a receiver for receiving the sensor data transmitted by the transmitter and a dosage determination unit for specifying a dosage of a V1a/V2 vasopressin receptor antagonist for the person on the basis of the sensor data and/or a treatment-duration determination unit for specifying the duration of the therapy with the V1a/V2 vasopressin receptor antagonist on the basis of the sensor data.

In a preferred embodiment, the sensor and the transmitter are components of a single device, the sensor unit. The sensor unit usually comprises a control unit for controlling the capture of measurement values and transmission of measurement values. The sensor unit usually comprises an energy supply in order to supply the sensor, the transmitter, the control unit and possibly further components of the sensor unit with energy, preferably with electrical energy. It is conceivable that the sensor unit has input means and/or output means in order to allow operation by a human user.

In a preferred embodiment, the receiver and the dosage determination unit and/or the treatment-duration determination unit are components of a single device, the evaluation unit, which device is not identical to the sensor unit. The evaluation unit can, for example, be a commercially available computer configured for dosage determination and/or treatment-duration determination. Such a computer can, for example, be a workstation computer (personal computer, PC for short) which is used for working on a screen. Another possibility is a mobile device such as a tablet computer, a smartphone, a laptop, a smartwatch or the like.

The evaluation unit receives sensor data from the sensor unit by means of the receiver and can determine a dosage and/or the duration of a treatment. If a person who is suffering from heart failure is not yet being therapied with a V1a/V2 vasopressin receptor antagonist, the evaluation unit can, on the basis of the transmitted sensor data, check whether the person's tissue water content has already normalized. For example, the evaluation unit can check whether a defined value (the critical tissue water content value) has been fallen short of or not after a defined time span (recovery time span) has passed. If the value has been fallen short of, a therapy with a V1a/V2 vasopressin receptor antagonist is not necessary; the specified dosage is “zero”. If the value has not been fallen short of, a therapy with a V1a/V2 vasopressin receptor antagonist is recommended and the dosage with a V1a/V2 vasopressin receptor antagonist is not equal to “zero”. If a person who is suffering from a heart failure is treated or is to be treated with a V1a/V2 vasopressin receptor antagonist, the dosage determination unit of the evaluation unit can ascertain a dosage on the basis of the received sensor data and possibly on the basis of further patient-specific data. The treatment-duration determination unit of the evaluation unit can ascertain a duration of the therapy with a V1a/V2 vasopressin receptor antagonist on the basis of the received sensor data and possibly on the basis of further patient-specific data.

Examples of patient data which can be used for ascertaining a dosage and/or duration of treatment are: sex, age, weight, weight change, height, severity of heart failure (e.g., in the form of a classification into classes NYHA I to IV), anamnesis, type and duration and amount of medicaments already taken, blood pressure, central venous pressure, respiratory rate, dyspnea, comorbidities and the like.

It is conceivable that the evaluation unit displays the determined (specified) dosage and/or the determined (specified) duration of treatment on a screen and/or saves it/them in a database and/or transmits it/them to another computer, for example that of the treating physician or other medical personnel.

In one embodiment of the present invention, the determined (specified) dosage is transmitted to a dosage system which automatically provides the person who is suffering from heart failure with the appropriate dosage. Such a dosage system can, for example, be a system which automatically administers the appropriate dosage to the person (a defined amount of a defined medicament at defined time points). However, another possibility is a system which reminds the person at defined time points (e.g., by means of an acoustic signal and/or visual signal and/or haptic signal) to take a defined amount of a defined medicament; moreover, the system can be configured such that it provides the person with the defined amount (e.g., by ejecting a defined amount of tablets from a container).

If the treatment of the patient with a V1a/V2 vasopressin receptor antagonist is successful and the tissue water content value reaches a value within a defined range (normal range), the dosage is set to “zero”, i.e., the treatment with the V1a/V2 vasopressin receptor antagonist is ended.

In a preferred embodiment, a person who is suffering from heart failure is admitted to a hospital. There, the person is equipped with a sensor for determining the tissue water content in the region of the chest. A physician and/or a caregiver inputs patient data of the person into an evaluation unit which can receive sensor data from the sensor. Patient data can also be captured automatically by means of one or more sensors. The evaluation unit is configured such that it calculates a recovery time span and/or a critical tissue water content value and/or a normal range for the tissue water content value on the basis of the patient data and/or the sensor data. The calculation can, for example, be made on the basis of an expert system. An expert system is a computer program which can, as an expert, assist people in solving relatively complex problems by deriving recommendations for action from a knowledge base. So-called If-Then relationships make it possible for human knowledge (connections in the world) to be implemented on a computer and to be thus made widely available (knowledge base). The If-Then relationships can, for example, be worked out through interviews with experts. However, it is also conceivable to use statistical methods and/or artificial intelligence methods in order, first, to proceed from a quantity of data from patients who are suffering from heart failure and to find correlations between the data, the severity of the heart failure and the success in therapy when taking certain measures, in order, then, to use said correlations in a further step for predicting and for ascertaining recommendations for action (e.g., calculating the optimal dosage). The evaluation unit continuously receives sensor data and monitors the tissue water content in the person. If necessary, critical tissue water content value and/or recovery time span and/or normal range are attuned to the course of the disease. If there is no fall in the tissue water content below the critical tissue water content value within the recovery time span, a message that a V1a/V2 vasopressin receptor antagonist should be administered to the person is output by the evaluation unit or transmitted thereby to another computer. The (optimal) dosage and/or the duration of treatment can be calculated by the evaluation unit on the basis of the sensor data and possibly on the basis of further patient data. The result of the calculations can be displayed on a screen, stored in a database, transmitted to a separate computer and/or transmitted to a dosage system (dispenser) for medicaments. The dispenser can be designed such that it automatically administers the appropriate dosage to the person over the duration of treatment or outputs the appropriate dosage over the duration of treatment, with the result that the person can personally take the dosage or a physician or caregiver can administer the dosage to the person. If the treatment with the V1a/V2 vasopressin receptor antagonist has ended, as indicated by the fact that either the end of the specified duration of treatment has been reached or the tissue water content has reached the normal range, the dosage is set to zero.

The invention is more particularly elucidated below with reference to FIGS. 1-6, showing example embodiments of features and combinations of features.

The same reference signs in FIGS. 1, 2 and 3 stand for the same components.

In the embodiment shown in FIG. 1, the system comprises two separate devices, a sensor unit (10) and an evaluation unit (20). The sensor unit (10) comprises a sensor (12) for capturing measurement values, on the basis of which the tissue water content in the chest region of a person can be ascertained, a transmitter unit (13), by means of which measurement values can be transmitted to the evaluation unit (20), and a control unit (11) for controlling the capture of measurement values and transmission of measurement values. The evaluation unit (20) comprises a receiving unit (24) for receiving measurement values from the sensor unit (10) and a dosage determination and/or treatment-duration determination unit (25). The evaluation unit (20) further comprises a transmission unit (23) for transmitting data and a control unit (21) for controlling the reception of measurement values via the receiving unit (24), for controlling the transmission of data by means of the transmission unit (23) and for controlling the components of the evaluation unit (20) and the data flows and signal flows between said components. The evaluation unit (20) further comprises an input and output unit (26) for control of the evaluation unit (20) by a user and/or for communication of the evaluation unit (20) with a user. For example, a keyboard, a mouse, a microphone, a touchscreen and the like can be considered as input means; for example, a screen, a printer, a loudspeaker, a data memory and the like are conceivable as output means.

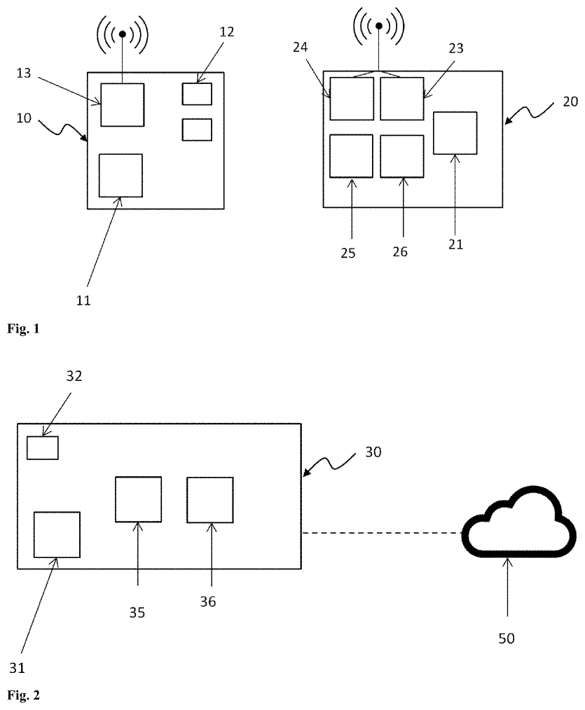

The embodiment of the system according to the invention that is shown in FIG. 2 comprises a single device: a combined sensor and evaluation unit (30). The combined sensor and evaluation unit (30) has a sensor (32), a control unit (31), a dosage determination and/or treatment-duration determination unit (35) and an input and output unit (36). The combined sensor and evaluation unit (30) is connected to further computer systems (represented by a cloud 50) via a network (represented by the dashed line).

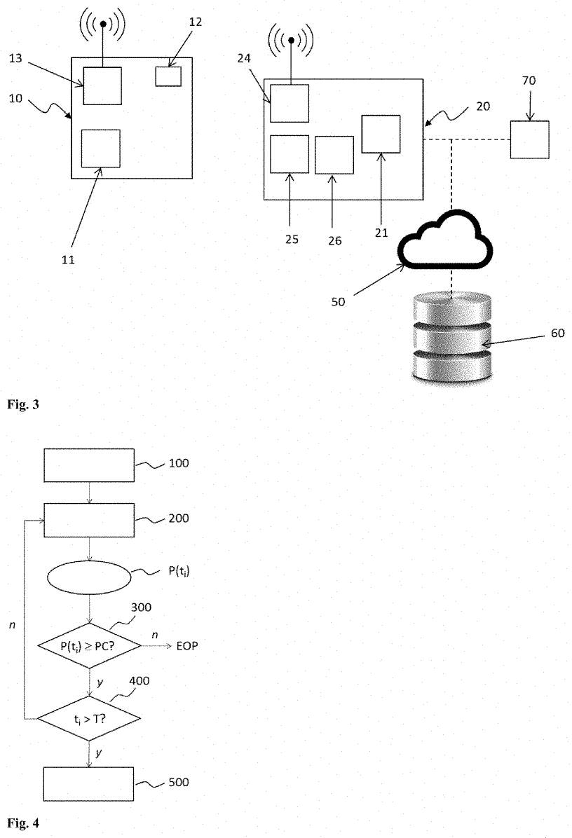

The embodiment of the system according to the invention that is shown in FIG. 3 comprises two separate devices: a sensor unit (10) and an evaluation unit (20).

The sensor unit (10) comprises a sensor (12) for capturing measurement values, on the basis of which the tissue water content in the chest region of a person can be ascertained, a transmitter unit (13), by means of which measurement values can be transmitted to the evaluation unit (20), and a control unit (11) for controlling the capture of measurement values and transmission of measurement values. The evaluation unit (20) comprises a receiving unit (24) for receiving measurement values from the sensor unit (10), a dosage determination and/or treatment-duration determination unit (25), a control unit (21) and input and output unit (26).

The evaluation unit (20) is connected to further computer systems (represented by a cloud 50), especially to a database (60), via a network (represented by the dashed line).

Patient data can be stored in the database. The patient data can have been input into the database, possibly via a network, by a physician and/or care personnel and/or the patient himself/herself and/or relatives.

However, patient data can also be/have been captured automatically by one or more further sensors.

The evaluation unit (20) is furthermore connected to a dispenser (70) for medicaments that contains multiple doses of a V1a/V2 vasopressin receptor antagonist. The evaluation unit (20) can transmit a dosage to the dispenser (70), with the result that the dispenser (70) can administer and/or provide the appropriate dosage.

FIG. 4 depicts a preferred embodiment of the method according to the invention as a flow chart.

In a first step (100), a sensor for determining a tissue water content in the region of the chest of a person is provided.

In a subsequent second step (200), the sensor provided is used for capturing data relating to the tissue water content in the region of the chest of the person. The person is suffering from heart failure and is being subjected to a standard therapy. Measurement values are captured at defined time points t₁, t₂, . . . t_{N}, from which the tissue water content at the respective time points can be ascertained. For each time point tᵢ, there is thus a tissue water content value P(tᵢ).

In a subsequent third step (300), a check is made as to whether the respective tissue water content value P(tᵢ) is greater than or equal to a critical tissue water content value PC. If the respective tissue water content value P(tᵢ) is below the critical tissue water content value PC (“n”), the person is responding to the standard therapy and no further measures are required (EOP: end of process). If the respective tissue water content value P(tᵢ) is greater than or equal to the critical tissue water content value PC, a check is made in a subsequent step (400) as to whether the recovery time span has already been exceeded (tᵢ>T?). If the recovery time span has not yet been exceeded (“n”), there is still hope that the standard therapy will work. If the recovery time span has been reached (“y”), the treatment of the patient with a V1a/V2 vasopressin receptor antagonist is recommended and/or initiated in a subsequent step (500). In this connection, the dosage with the V1a/V2 vasopressin receptor antagonist and/or the duration of the treatment can be specified on the basis of the last-ascertained tissue water content value, on the basis of multiple ascertained tissue water content values or on the basis of the entire time course of ascertained tissue water content values.

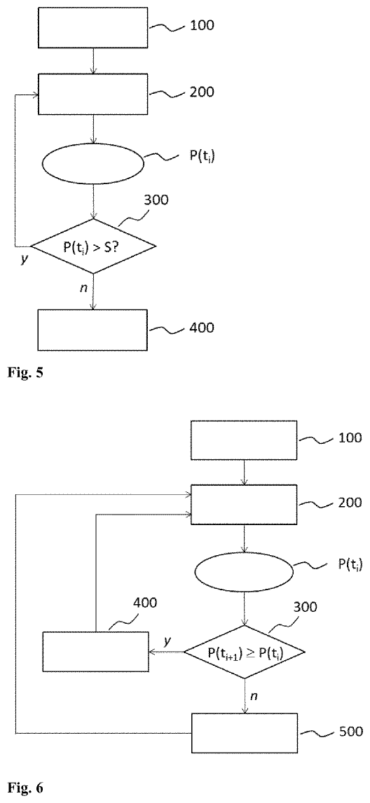

FIG. 5 depicts a further preferred embodiment of the method according to the invention as a flow chart.

In a first step (100), a sensor for determining a tissue water content in the region of the chest of a person is provided.

In a subsequent second step (200), the sensor provided is used for capturing data relating to the tissue water content in the region of the chest of the person. The person is suffering from heart failure and is being treated with a V1a/V2 vasopressin receptor antagonist. Measurement values are captured at defined time points tᵢ, t₂, . . . t_{N}, from which the tissue water content at the respective time points can be ascertained. For each time point tᵢ (i=1, 2, . . . N), there is thus a tissue water content value P(tᵢ).

In a subsequent third step (300), a check is made as to whether the respective tissue water content value P(tᵢ) is already in a normal range or whether it is outside the normal range. The normal range can, for example, be characterized by a threshold value S. If the tissue water content value is greater than the threshold value S (“y”), then it is outside the normal range and the therapy with the V1a/V2 vasopressin receptor antagonist is continued. If the respective tissue water content value P(tᵢ) is not above the threshold value S (“n”), the therapy with the V1a/V2 vasopressin receptor antagonist is successful. In a subsequent step (400), the further dosage with the V1a/V2 vasopressin receptor antagonist is attuned to the success of the therapy; for example, it can be reduced or set to “zero”.

FIG. 6 depicts a further preferred embodiment of the method according to the invention as a flow chart.

In a first step (100), a sensor for determining a tissue water content in the region of the chest of a person is provided.

In a subsequent second step (200), the sensor provided is used for capturing data relating to the tissue water content in the region of the chest of the person. The person is suffering from heart failure and is being treated with a V1a/V2 vasopressin receptor antagonist. Measurement values are captured at defined time points tᵢ, t₂, . . . t_{N}, from which the tissue water content at the respective time points can be ascertained. For each time point tᵢ (i=1, 2, . . . N), there is thus a tissue water content value P(tᵢ).

In a subsequent third step (300), a check is made as to whether the tissue water content is rising (P(tᵢ₊₁)>P(tᵢ)), falling (P(tᵢ₊₁)<P(tᵢ)) or remaining unchanged (P(tᵢ₊₁)=P(tᵢ)) over the course of time. If it should not be falling, a higher dosage can be recommended and/or administered in a further step (400). If it should be falling, a lower dosage can be recommended and/or administered in a further step (500).

It is conceivable to combine together all or some of the sequences of events that are shown in FIGS. 4, 5 and/or 6.

1-3. (canceled)

4: A system for treating cardiac insufficiency, comprising;
a sensor configured to capture data relating to the tissue water content in the body of a person in the region of the chest;
a transmitter configured to transmit the captured sensor data to a receiver;
the receiver configured to receive the sensor data transmitted by the transmitter; and
a dosage determination unit configured to determine a dosage of a V1a/V2 vasopressin receptor antagonist for a therapy for the person based on the received sensor data.

5: The system of claim 4, wherein the dosage determination unit is configured to:
determining, based on the sensor data, whether the tissue water content falls short of a critical tissue water content value within a recovery time span; and
in response to determining that tissue water content is not less than the critical tissue water content value, recommend the therapy with the V1a/V2 vasopressin receptor antagonist.

6: The system of claim 5, wherein the dosage determination unit is configured to calculate the critical tissue water content value and the recovery time span based on the sensor data or based on further patient data.

7-8. (canceled)

9: A non-transitory computer-readable storage medium comprising one or more programs for treating cardia insufficiency, wherein the one or more programs, when executed by one or more processors, cause the one or more processors to:
receive sensor data from a sensor, the sensor data relating to a tissue water content in the chest region of a person; and
determine a dosage of a V1a/V2 vasopressin receptor antagonist for treating the person based on the sensor data.

10: The non-transitory computer-readable storage medium of claim 9, wherein the one or more processors are caused to:
determine, based on the sensor data, whether the tissue water content falls short of a critical tissue water content value within a recovery time span and
in response to determining that tissue water content is not less than the critical tissue water content value, recommend the therapy with the V1a/V2 vasopressin receptor antagonist.

11: The non-transitory computer-readable storage medium of claim 10, wherein the one or more processors are caused to calculate the critical tissue water content value or the recovery time span based on the sensor data or based on further patient data.

12. (canceled)

13: A method, comprising:
providing a sensor for determining a tissue water content in the region of the chest of a person suffering from heart failure;
capturing, by the sensor, data relating to the tissue water content in the region of the chest of the person,
determining a dosage of a V1a/V2 vasopressin receptor antagonist for a therapy to treat the person based on the sensor data relating to the tissue water content.

14: The method of claim 13, comprising:
determining, based on the sensor data, whether the tissue water content falls short of a critical tissue water content value within a recovery time span; and
in response to determining that the tissue water content is not less than the critical tissue water content value, recommend the therapy with the V1a/V2 vasopressin receptor antagonist.

15: The method of claim 14, further comprising:
determining the critical tissue water content value and the recovery time span based on the sensor data or based on further patient data.

16: The method of claim 13, further comprising:
transmitting the determined dosage to a dispenser for medicaments containing multiple doses of the V1a/V2 vasopressin receptor antagonist.

17: The method of claim 13, further comprising:
determining a duration of the treatment with the V1a/V2 vasopressin receptor antagonist based on the sensor data.

18: The system of claim 4, comprising:
a treatment-duration determination unit configured to specify a duration of the therapy with the V1a/V2 vasopressin receptor antagonist.

19: The system of claim 18, wherein the treatment-duration determination unit is configured to:
determining, based on the sensor data, whether the tissue water content falls short of a critical tissue water content value within a recovery time span; and
in response to determining that the critical tissue water content value does not short of the critical tissue water content value, recommend the therapy with the V1a/V2 vasopressin receptor antagonist.

20: The system of claim 18, wherein the treatment-duration determination unit is configured to calculate the duration of treatment with the V1a/V2 vasopressin receptor antagonist based on the sensor data.

21: The system of claim 19, wherein the treatment-duration determination unit is configured to calculate the critical tissue water content value and the recovery time span based on the sensor data or based on further patient data.

22: The non-transitory computer-readable storage medium of claim 9, wherein the one or more processors are caused to:
determine a duration of treatment with the V1a/V2 vasopressin receptor antagonist based on the sensor data.
